(19) United States
(12) Patent Application Publication
(10) Pub. No.: US 2012/0259132 A1
(43) Pub. Date: Oct. 11, 2012

(54) PROCESS TO PREPARE AN ETHANOL-DERIVATE

(75) Inventors: Meindert Jan Lippits, (US); Bernard Egbert Nieuwenhuijs, (US)

(73) Assignee: Universiteit Leiden

(21) Appl. No.: 13/440,323

(22) Filed: Apr. 5, 2012

Related U.S. Application Data
(60) Provisional application No. 61/472,390, filed on Apr. 6, 2011.

(51) Int. Cl. C07C 1/20; C07C 41/14; C07C 27/00; B01J 37/02; C07C 209/00; B01J 23/02; B01J 23/72; C07D 301/22; C07C 41/02

(52) U.S. Cl. 549/518; 568/671; 568/867; 568/680; 564/487; 585/639; 502/340; 585/640; 502/304; 977/773; 977/902; 977/811; 977/810

(57) ABSTRACT

The invention is directed to a process to prepare an ethanol-derivate compound or compounds by reacting ethanol in the presence of molecular oxygen and a catalyst comprising a gamma-alumina carrier, metal nano-particles wherein the metal is selected from silver, copper or gold. The invention is also directed to processes to prepare such a catalyst.

### CROSS-REFERENCE TO RELATED APPLICATIONS

This application claims the benefit of U.S. Provisional Application No. 61/472,390, filed Apr. 6, 2011, which is hereby incorporated herein in its entirety by reference.

### FIELD OF THE INVENTION

The invention is directed to prepare an ethanol-derivate. The invention is especially related to prepare ethylene oxide, diethyl ether or ethylene or any mixtures comprising these ethanol-derivatives.

### BACKGROUND OF THE INVENTION

Processes to prepare ethylene from ethanol is described in U.S. Pat. No. 4,847,223. In this process ethanol in admixture with water is reacted to ethylene in the presence of a ZSM-5 containing catalyst onto which triflic acid has been incorporated.

EP-A-1792885 describes a process to convert ethanol into ethylene in the presence of a heterogeneous catalyst consisting of a heteropolyacid.

EP-A-1861196 describes a process for preparing ethylene oxide by epoxidation of ethylene with oxygen using a silver based catalyst. The ethylene oxide may be converted to ethylene glycol, ethylene glycol ether or ethanol amine according to this publication.

Ethanol is an interesting feedstock in that it can be prepared from various sources of biomass. There is a widespread interest to develop processes to prepare various chemical products from ethanol. The present invention is in particular directed to a novel process to prepare ethylene, diethyl ether and/or ethylene oxide directly from ethanol.

### BRIEF SUMMARY OF THE INVENTION

This object is achieved by the following process. Process to prepare an ethanol-derivate compound or compounds by reacting ethanol in the presence of molecular oxygen and a catalyst comprising a gamma-alumina carrier, metal nano-particles wherein the metal is selected from silver, copper or gold.

Applicants found that with the above process desirable ethanol-derivates can be obtained in a high selectivity and yield. Furthermore the process may be performed at relatively low pressures. Additional advantages shall be discussed below when discussing the various preferred embodiments of the invention.

The invention is also directed to a process to prepare an ethanol-derivate compound or compounds by reacting ethanol in the presence of a catalyst comprising a gamma-alumina carrier, silver metal nano-particles having an average size of less than 5 nm as determined by XRD and an additive selected from the group of a cerium compound or an alkaline metal compound selected from the group consisting of Na, Li or K.

The invention is also directed to a first process to prepare a catalyst composition comprising a gamma-alumina carrier, metal nano-particles, wherein the metal is selected from silver, copper or gold, and an additive selected from the group of an alkaline metal compound as present as an oxide or hydroxide of the alkaline metal wherein:

in a first catalyst preparation step a gamma-alumina carrier is contacted with an aqueous solution comprising a salt of the alkaline metal in an impregnation step to obtain a loaded alumina carrier, and wherein the weight of alkaline metal as deposited on the alumina surface of the alumina carrier is greater than the weight of alkaline metal as present in the final catalyst composition, drying the loaded alumina carrier and subjecting the dried loaded alumina carrier to a calcination step,

loading the silver, copper or gold metal to the calcined loaded carrier in a second catalyst preparation step by contacting with an aqueous solution comprising a silver, copper or gold metal salt and drying to obtain the catalyst composition.

The invention is also directed to a second process to prepare a catalyst composition comprising a gamma-alumina carrier, metal nano-particles wherein the metal is selected from silver, copper or gold and an additive selected from the group of cerium compound or an alkaline metal compound as present as an oxide or hydroxide of the cerium or alkaline metal wherein:

in a first catalyst preparation step a gamma-alumina carrier is contacted with an aqueous solution comprising a salt of the cerium or alkaline metal in an impregnation step to obtain a loaded alumina carrier, drying the loaded alumina carrier and subjecting the dried loaded alumina carrier to a calcination step,

and loading the silver, copper or gold metal to the calcined loaded carrier in a second catalyst preparation step by contacting with an aqueous solution of a silver, copper or gold metal salt and drying to obtain the catalyst composition, wherein

the gamma-alumina carrier used to prepare the catalyst in the first catalyst preparation step has a dried surface which is expressed by its iso-electric point of greater than 7 as measured by preparing an aqueous slurry of the alumina at ambient conditions and measuring the pH of the water phase, wherein the iso-electric point is the measured pH.

The following embodiments are encompassed by the present invention:

1. Process to prepare an ethanol-derivate compound or compounds by reacting ethanol in the presence of molecular oxygen and a catalyst comprising a gamma-alumina carrier, metal nano-particles wherein the metal is selected from silver, copper or gold.

2. Process according to embodiment 1, wherein the catalyst comprises an additive selected from the group of a cerium compound or an alkaline metal compound.

3. Process according to embodiment 2, wherein the additive is an alkaline metal compound selected from the group consisting of Na, Li or K.

4. Process according to embodiment 3, wherein the alkaline metal compound is Li and wherein the Li metal is present in the catalyst as an oxide or hydroxide.

5. Process according to any one of embodiments 1-4, wherein the gamma-alumina carrier used to prepare the catalyst comprised between 0.05 and 0.2 wt % of sodium oxide and between 0.01 and 0.1 wt % of an iron oxide.

6. Process according to any one of embodiments 1-4, wherein the metal nano-particles have an average size of less than 5 nm as determined by XRD.

7. Process according to any one of embodiments 1-4, wherein the nano-particles are gold nano-particles.

8. Process according to any one of embodiments 1-4, wherein the prepared ethanol-derivative is ethylene and wherein the nano-particles are copper nano-particles.

9. Process according to embodiment 8, wherein the temperature is between 350 and 450° C.

10. Process according to any one of embodiments 1-4, wherein the ethanol-derivative compounds are ethylene oxide, diethyl ether and/or ethylene.

11. Process according to any one of embodiments 1-4, wherein the molar ratio of ethanol and molecular oxygen is between 1:0.5 and 1:10.

12. Process according to any one of embodiments 1-4, wherein the pressure at which the process is performed is between 0.1 and 1 MPa and the gas hourly space velocity (GHSV) is in the range of from 500 to 5000 h⁻¹.

13. Process according to embodiment 12, wherein the catalyst comprises nano-particles of an average size of less than 5 nm, the additive is Li₂O and the temperature is between 100 and 250° C.

14. Process according to any one of embodiments 1-4, wherein the process is performed in a reactor comprising the catalyst, to which reactor a gaseous feed comprising ethanol, oxygen and an optional diluting gas is supplied and from which reactor an effluent is discharged comprising the ethanol-derivative compound or compounds, oxygen and the optional diluting gas.

15. Process according to embodiment 14, wherein the reactor is a fluidized bed reactor.

16. Process according to embodiment 14, wherein the reactor is a packed bed reactor.

17. Process according to embodiment 14, wherein from the effluent any non-converted ethanol is separated from the reactor effluent and recycled to the feed comprises less than 10 vol % water.

18. Process according to embodiment 17, wherein ethanol is separated from the reactor effluent by means of distillation.

19. Process according to embodiment 14, wherein carbon dioxide as present in the reactor effluent is recycled to the reactor to act as diluent for the ethanol feed.

20. Process to prepare ethylene glycol, an ethylene glycol ether or an ethanol amine from ethanol by first preparing ethylene oxide according to the process according to claim 10 and converting ethylene oxide as obtained into the desired ethylene glycol, an ethylene glycol ether or an ethanol amine.

21. Process to prepare a catalyst composition comprising a gamma-alumina carrier, metal nano-particles, wherein the metal is selected from silver, copper or gold, and an additive selected from the group of an alkaline metal compound as present as an oxide or hydroxide of the alkaline metal wherein:

in a first catalyst preparation step a gamma-alumina carrier is contacted with an aqueous solution comprising a salt of the alkaline metal in an impregnation step to obtain a loaded alumina carrier, and wherein the weight of alkaline metal as deposited on the alumina surface of the alumina carrier is greater than the weight of alkaline metal as present in the final catalyst composition, drying the loaded alumina carrier and subjecting the dried loaded alumina carrier to a calcination step,

loading the silver, copper or gold metal to the calcined loaded carrier in a second catalyst preparation step by contacting with an aqueous solution comprising a silver, copper or gold metal salt and drying to obtain the catalyst composition.

22. Process according to embodiment 21, wherein pore impregnation is applied in the first catalyst preparation step and wherein the weight of the alkaline metal as present in the aqueous solution is larger than the weight of alkaline metal as present in the final catalyst composition.

23. Process according to claim any one of embodiments 21-22, wherein in the second catalyst preparation step the Ag, Au or Cu metal is added to the catalyst via a homogenous deposition precipitation process.

24. Process according to embodiment 23, wherein the homogenous deposition precipitation process uses urea or an alkali carbonate as the precipitating agent.

25. Process according to embodiment 24, wherein the precipitating agent is added to a slurry of the calcined and loaded alumina carrier and the aqueous solution of the silver, copper or gold metal salt at a temperature of between 20 and 80° C.

26. Process according to embodiment 25, wherein the slurry is allowed to cool down when the pH of the slurry reaches 8 or above.

27. Process according to any one of embodiments 21-22, wherein the gamma-alumina carrier used to prepare the catalyst has a dried surface which is expressed by its iso-electric point of greater than 7 as measured by preparing an aqueous slurry of the alumina at ambient conditions and measuring the pH of the water phase, wherein the iso-electric point is the measured pH.

28. Process according to any one of embodiments 21-22, wherein the γ-alumina carrier used to prepare the catalyst comprised between 0.05 and 0.2 wt % of sodium oxide and between 0.01 and 0.1 wt % of an iron oxide.

29. Process according to any one of embodiments 21-22, wherein the metal nano-particles have an average size of less than 5 nm as determined by XRD.

30. Process according to any one of embodiments 21-22, wherein the additive is an alkaline metal compound selected from the group consisting of Na, Li or K.

31. Process according to embodiment 30, wherein the alkaline metal compound is Li.

32. Process to prepare a catalyst composition comprising a gamma-alumina carrier, metal nano-particles wherein the metal is selected from silver, copper or gold and an additive selected from the group of cerium compound or an alkaline metal compound as present as an oxide or hydroxide of the cerium or alkaline metal wherein:

in a first catalyst preparation step a gamma-alumina carrier is contacted with an aqueous solution comprising a salt of the cerium or alkaline metal in an impregnation step to obtain a loaded alumina carrier, drying the loaded alumina carrier and subjecting the dried loaded alumina carrier to a calcination step,

and loading the silver, copper or gold metal to the calcined loaded carrier in a second catalyst preparation step by contacting with an aqueous solution of a silver, copper or gold metal salt and drying to obtain the catalyst composition, wherein

the gamma-alumina carrier used to prepare the catalyst in the first catalyst preparation step has a dried surface which is expressed by its iso-electric point of greater than 7 as measured by preparing an aqueous slurry of the alumina at ambient conditions and measuring the pH of the water phase, wherein the iso-electric point is the measured pH.

33. Process according to embodiment 32, wherein in the second catalyst preparation step the Ag, Au or Cu metal is added to the catalyst via a homogenous deposition precipitation process.

34. Process according to embodiment 33, wherein the homogenous deposition precipitation process uses urea or an alkali carbonate as the precipitating agent.

35. Process according to any one of embodiments 33-34, wherein the precipitating agent is added to a slurry of the calcined and loaded alumina carrier and the aqueous solution of the silver, copper or gold metal salt at a temperature of between 20 and 80° C.

36. Process according to embodiment 34, wherein the slurry is allowed to cool down when the pH of the slurry reaches 8 or above.

37. Process according to any one of embodiments 32-33, wherein the γ-alumina carrier used to prepare the catalyst comprised between 0.05 and 0.2 wt % of sodium oxide and between 0.01 and 0.1 wt % of an iron oxide.

38. Process according to any one of embodiments 32-33, wherein the metal nano-particles have an average size of less than 5 nm as determined by XRD.

39. Process according to any one of embodiments 32-33, wherein the additive is an alkaline metal compound selected from the group consisting of Na, Li or K.

40. Process according to embodiment 39, wherein the alkaline metal compound is Li.

### BRIEF DESCRIPTION OF THE DRAWINGS

FIG. 1 is the ethylene oxide selectivity for various catalysts as a function of temperature.

FIG. 2 is the diethyl ether selectivity for various catalysts as a function of temperature.

FIG. 3 is the ethylene selectivity for various catalysts as a function of temperature.

FIG. 4 is the ethylene oxide and carbon dioxide selectivity for a catalyst as a function of temperature.

FIG. 5 shows the ethanol conversion at different temperatures for different copper based catalysts.

### DETAILED DESCRIPTION OF THE INVENTION

The ethanol feedstock may be chemically prepared, for example from synthesis gas, i.e. a mixture of carbon monoxide and hydrogen or derived from biomass, i.e. so-called bio-ethanol. An example of bio-ethanol is ethanol produced by the fermentation of corn or sugar cane. Other sources for preparing bio-ethanol are non-food biomass sources such a cellulose or algae.

Applicants have found that the process according to the invention can be used to prepare a wide variety of ethanol derivates like ethylene oxide, diethyl ether or ethylene or any mixtures comprising the ethanol-derivatives. Processes which are performed in the presence of oxygen have found to yield ethylene oxide or ethylene as a major product while maintaining a low level of CO and CO₂ formation. Examples of by-products that are formed are di-ethyl ether which is a valuable by-product in its own right. Di-ethyl ether can be isolated and used as fuel component, for example in an aviation fuel composition or in a diesel formulation.

The molar ratio of ethanol and molecular oxygen is preferably between 1:0.5 and 1:10. A higher oxygen content is not advantageous because the selectivity to the desired ethanol-derivative compounds will be lower and more carbon dioxide will be formed. Lower oxygen content will result in coke formation and catalyst deactivation. The temperature is preferably between 100 and 450° C. The oxygen may be diluted with a gas, such as argon, helium, nitrogen or carbon dioxide. The oxygen may also be present as part of air or enriched air or diluted air, for example air diluted with argon, helium, nitrogen or carbon dioxide. Nitrogen or carbon dioxide is preferred as diluting gas. The mixture of oxygen and ethanol and an optional diluting gas or gasses may be suitably obtained by contacting gaseous oxygen with liquid ethanol, suitably by bubbling the gaseous oxygen through liquid ethanol. The ethanol will evaporate into the oxygen bubbles to obtain the desired oxygen/ethanol mixture. The gaseous oxygen may be diluted with the diluting gas or gasses or alternatively the diluting gas or gasses are added after contacting the gaseous oxygen with the liquid ethanol. The pressure at which the process is performed is preferably between 0.1 and 1 MPa. The gas hourly space velocities (GHSV) are suitably in the range of from 500 to 5000 h⁻¹.

The catalyst comprises a γ-alumina carrier (gamma-alumina; γ-Al₂O₃). The γ-alumina used to prepare the catalyst may comprise small amounts of metals. Applicants found that a suited catalyst can be prepared starting from a γ-alumina carrier comprising between 0.05 and 0.2 wt % of sodium oxide (calculated as Na₂O) and between 0.01 and 0.1 wt % of an iron oxide (calculated as Fe₂O₃).

The metal nano-particles preferably have an average size of below 10 nm and more preferably below 5 nm as determined by XRD. When the XRD technique does not detect particles an average particle size of below 3 nm is concluded. The presence of nano-particles can be confirmed using High Resolution TEM. The metal of the nano-particles is selected from silver, copper or gold. The content of copper in the catalyst is preferably between 0.1 and 5 wt %. The content of silver in the catalyst is preferably between 0.1 and 5 wt %. The content of gold in the catalyst is preferably between 0.5 and 10 wt %, more preferably between 0.5 and 6 wt %. The surface area of the catalyst is preferably between 250 and 275 m²/g.

The preference for a metal will depend on the desired ethanol-derivative to be prepared. Applicants found that ethylene as the ethanol derivative compound can be prepared in a high yield using a catalyst wherein the nano-particles are copper nano-particles. Preferably the temperature for this process is between 350 and 450° C. This process is advantageous because it uses a relatively simple catalyst, i.e. not containing any molecular sieves, and because of its high yield achievable at moderate operating pressures.

To prepare ethylene oxide as the ethanol-derivative compound it has been found advantageous to use a catalyst also comprising an additive selected from the group of a cerium compound or an alkaline metal compound. The cerium compound is preferably CeOₓ wherein x is 1, 2 or 1.5. Preferably the additive is an alkaline metal compound selected from the group consisting of Na, Li or K and more preferably Li. The alkaline metal compounds may be present in the catalyst as an oxide or hydroxide. Alkaline metal compound in the fresh catalyst, before use in the process of the present invention, will most likely be present as an oxide. The preferred Li metal compound will then be present as Li₂O. When reference is made to the content of said additives it is assumed that the Ce or alkaline metal is present in its oxide form. The content of these additives in the catalyst is preferably between 1 and 15 wt %. The preferred additive is Li₂O because for example processes using a Li₂O based catalyst according to the present invention have shown a high selectivity in the one step process to ethylene oxide. The gold, copper and silver based catalyst comprising also Li₂O are all suited to convert ethanol in a high yield at relatively low temperatures to ethylene oxide. The gold based catalyst is preferred because it has shown the highest activity and selectivity in our experiments.

Applicants have shown that a high selectivity and yield in a one step process to ethylene oxide is possible with a catalyst comprising either one of these metals and Li₂O as the additive. The fact that ethylene oxide can be prepared in a one step process from ethanol is very advantageous because it eliminates the need to first prepare ethylene as an intermediate as in the prior art processes. Further advantages are that the process is performed at relatively low temperatures and at low pressures. The temperature is preferably between 100 and 250° C.

The catalyst is preferably reduced before use. More preferably by contacting the catalyst with hydrogen, more preferably 4% hydrogen diluted in Helium or Argon at an elevated temperature of around 400° C. The catalyst may be regenerated after a period of use by removing carbon deposited on the catalyst by contacting the catalyst with a gaseous stream comprising an oxygenate, preferably oxygen at temperatures between 300 and 400° C.

The catalyst can have any form when used in the process according to the invention, like for example crushed particles, tablets or extrudates. The catalyst may also be present as a coating on a support or as a reactive layer on the interior of a conduit through which reactants are supplied. The catalyst comprising gold and its preparation is known and described in WO-A-2006/065138. Catalysts based on silver and copper and their preparation are known and described in Catalysis Today 145 15 Jul. 2009, pages 27-33.

Preferably the catalyst is prepared according to the first and/or second process to prepare a catalyst composition as described above. In the first process to prepare a catalyst composition it is preferred to dry the gamma-alumina before performing the first catalyst preparation step. Drying will result in a dry surface of the alumina carrier. In the second process to prepare a catalyst composition the gamma-alumina carrier has a dried surface. Drying may be performed by keeping the alumina at elevated temperatures, for example at a temperature between 80 and 200° C., for a certain period of time. The dried alumina suitable for use will preferably have a dried surface which may be expressed by its iso-electric point. The iso-electric point of the surface is measured by preparing an aqueous slurry of the alumina at ambient conditions and measuring the pH of the water phase. The measured pH is the iso-electric point of the alumina surface. The preferred dried gamma-alumina has an iso-electric point as measured according to this procedure of greater than 7 and more preferably between 7 and 8.

When the catalyst is prepared which also comprises an additive selected from the group of an alkaline metal compound according to the first process to prepare a catalyst composition the gamma-alumina is contacted in a first catalyst preparation step with an aqueous solution comprising a salt of the alkaline metal in an impregnation step, suitably a pore volume impregnation step. In this step the weight of alkaline metal as deposited on the alumina surface is greater than the weight of alkaline metal as present in the final catalyst composition. Preferably more than 10 wt % excess alkaline metal, and more preferably more than 50 wt % excess alkaline metal, is present on the surface of the intermediate catalyst composition. When pore volume impregnation is applied the weight of alkaline metal as present in the aqueous solution is thus greater than the weight alkaline metal as present in the final catalyst composition and preferably present in an excess of more than 10 wt %.

Catalyst composition having a cerium additive may be prepared according to the above procedures. However the amount of cerium used in the preparation, for example by means of a pore volume impregnation process, may be about the amount as present in the final catalyst composition.

Preferred salts of the cerium or alkaline metal compound suited to prepare the catalyst composition are soluble in water and decompose at the calcination conditions described below. Examples of salts which are suited are nitrates, such as for example LiNO₃, LiIO₃, LiI, LiMnO₄.3H₂O, LiNO₃.3H₂O, LiNO₂.2H₂O, Li₂SO₄, LiC₂H₃O₂.2H₂O, LiNH₂, LiHCO₃, LiC₆H₅O₇.4H₂O. After the water has evaporated in the impregnation step it is preferred to dry the catalyst for a prolonged period of time, preferably at a temperature of between 80 and 200° C. The optimal drying time can be easily established for the chosen temperature and catalyst and may be for example between 5 and 20 hours. After the drying step it is preferred to perform a calcination step, preferably at a temperature of above 300° C. in the presence of oxygen. In a second catalyst preparation step the Ag, Au or Cu metal is preferably added to the catalyst via a homogenous deposition precipitation process, preferably using urea or an alkali carbonate as the precipitating agent. In this process the desired amount of a salt of these metals as dissolved in water is contacted with the calcined and loaded alumina carrier as obtained in the first step. Suitable salts are nitrates, for example Cu(NO₃)₂.3aq, AgNO₃, and other in water soluble salts like for example chlorides, such as for example HAuCl₄.3aq or AuCl₃. Preferably the precipitating agent is added to the thus obtained slurry at a temperature of between 20 and 80° C. allowing the precipitating agent to decompose. The pH slowly increases and when the pH reaches 8 or above the slurry is allowed to cool down. The remaining aqueous solution is separated from the solid particles, for example by means of filtration, and the solid particles are preferably washed with water to remove any easily soluble salts such as chlorides and urea. Contacting with water should be minimized in order to avoid removal of the alkaline hydroxide or oxides as present on the catalyst surface. The thus obtained solid particles are subsequently dried to obtain the final catalyst. As stated above it is preferred to reduce the thus obtained catalyst before actual use. Applicants found that it is not required to perform a calcination step after performing this second catalyst preparation step and before performing reducing the catalyst. Calcination in the context of the present invention is any process wherein the catalyst is subjected to a thermal treatment at a temperature of above 250° C. in the presence of gaseous oxygen.

Contacting the ethanol with the catalyst may be performed in any type of reactor comprising the catalyst and suited for contacting the gaseous feed with the heterogeneous catalyst. The process is performed in a reactor comprising the catalyst, to which reactor a gaseous feed comprising ethanol, oxygen and preferably a diluting gas is supplied and from which reactor an effluent is discharged comprising the ethanol-derivative compound or compounds, oxygen and the optional diluting gas. Examples of suitable reactors are fluidized bed reactors and packed bed reactors. Fluidized bed reactors are advantageous because catalyst can be more easily regenerated to remove any carbon deposits on the catalyst and the temperature in the reactor can be easily regulated to be within the desired temperature range. Packed bed reactors are advantageous because the catalyst will be less exposed to attrition as will be the case in a fluidized bed reactor. Preferred packed bed reactors are single tubular or multi-tubular reactors. The reaction is exothermic and cooling is suitably applied to maintain a temperature in the range suited for achieving a high selectivity to the desired ethanol-derivative compound. Cooling can be achieved by external cooling the conduit containing the catalyst or by internal cooling by dilution of the ethanol/oxygen feed with a gas, like for example the earlier listed dilution gases argon, helium, nitrogen or carbon dioxide. External cooling can be evaporating water. Catalysts may also be present as a coating on the interior of the reactor, for example coated on a network which is fixed in the reactor or on the inside of the reactor transport conduits, like in a micro-channel reactor, as for example described in WO-A-2010009021 or in a monolith type reactor.

To achieve the highest yield to the desired products it may be advantageous to convert only part of the ethanol when contacting ethanol with the catalyst and recycling any non-converted ethanol to the reactor. In this process ethanol will be separated from the effluent of the reactor, preferably by means of distillation. Preferably the ethanol which is recycled to the feed of the reactor comprises less than 10 vol % water. This to avoid a build-up of water which is disadvantageous for the catalyst stability. Preferably oxygen is also recycled to the reactor. Carbon dioxide is one of the by-products of the present process as present in the reactor effluent. In a preferred embodiment of the invention carbon dioxide is recycled to the reactor to act as diluent for the ethanol feed.

The ethylene oxide as prepared in the above process may be advantageously further converted into ethylene glycol, an ethylene glycol ether or an ethanol amine. The conversion into ethylene glycol or the ethylene glycol ether may comprise, for example, reacting the ethylene oxide with water, suitably using an acidic or a basic catalyst. Suitably the gaseous effluent of the reactor in which the ethylene oxide is formed, as described above, can be directly contacted with such an aqueous solution, for example an aqueous solution containing sodium hydroxide, in a process to prepare ethylene glycol. In another process for making predominantly the ethylene glycol and less ethylene glycol ether, the ethylene oxide may be reacted with a ten fold molar excess of water, in a liquid phase reaction in presence of an acid catalyst, e.g. 0.5-1.0% w sulphuric acid, based on the total reaction mixture, at 50-70° C. at 100 kPa absolute, or in a gas phase reaction at 130-240° C. and 2000−4000 kPa absolute, preferably in the absence of a catalyst. If the proportion of water is lowered the proportion of ethylene glycol ethers in the reaction mixture is increased. The ethylene glycol ethers thus produced may be a di-ether, tri-ether, tetra-ether or a subsequent ether. Alternative ethylene glycol ethers may be prepared by converting the ethylene oxide with an alcohol, in particular a primary alcohol, such as methanol or ethanol, by replacing at least a portion of the water by the alcohol. The ethylene oxide may be converted into ethylene glycol by first converting the ethylene oxide into ethylene carbonate by reacting with carbon dioxide, and subsequently hydrolyzing the ethylene carbonate to form ethylene glycol. For applicable methods, reference is made to U.S. Pat. No. 6,080,897, which is incorporated herein by reference. The conversion into the ethanol amine may comprise reacting ethylene oxide with an amine, such as ammonia, an alkyl amine or a dialkyl amine. Anhydrous or aqueous ammonia may be used. Anhydrous ammonia is typically used to favour the production of mono ethanol amine. For methods applicable in the conversion of ethylene oxide into the ethanol amine, reference may be made to, for example U.S. Pat. No. 4,845,296, which is incorporated herein by reference.

Ethylene glycol and ethylene glycol ethers may be used in a large variety of industrial applications, for example in the fields of food, beverages, tobacco, cosmetics, thermoplastic polymers, curable resin systems, detergents, heat transfer systems, etc. Ethanol amines may be used, for example, in the treating (“sweetening”) of natural gas.

The invention is thus also directed to a process to prepare ethylene glycol, an ethylene glycol ether or an ethanol amine from ethanol by first preparing ethylene oxide according to the process described above and converting ethylene oxide as obtained into the desired ethylene glycol, an ethylene glycol ether or an ethanol amine.

The invention shall be illustrated by the following non-limiting examples.

### EXAMPLES

### Example 1

The gold comprising catalysts also comprising ceria (denoted as CeOₓ) and/or Li₂O used in the experiments were prepared by pore volume impregnation of γ-Al₂O₃ (as obtained from BASF, De Meern (NL), sample code: Al-4172 Lot: PP10) with the corresponding nitrates.

The pH of an aqueous solution of 5 grams of the γ-Al₂O₃ in 50 ml Millipore water (18.2 MΩ cm resistive Milli-Q water) was 6.5.

The γ-Al₂O₃ was dried in a stove at 105° C. for 48 hours. The pH of an aqueous solution of 5 grams of the dried alumina in 50 ml Millipore water (18.2 MΩ cm resistive Milli-Q water) was 7.5. The dried alumina was used to prepare the catalyst.

The γ-Al₂O₃ was analysed by means of an XRF scan which showed that it contained ±0.05 wt % Na₂O, ±0.1 wt % SiO₂ and ±0.05 wt % Fe₂O₃.

When preparing the catalyst containing both CeOₓ and Li₂O first the CeOₓ was impregnated by means of pore impregnation followed by impregnation by means of impregnation of the Li₂O.

In the impregnation step 1 gram of the dried gamma-alumina was contacted with 10 ml of a aqueous solution of Ce nitrate salt. The weight of cerium added in the pore impregnation step as compared to the weight of cerium in the final catalyst was about the same.

In the pore impregnation step 1 gram of the dried gamma-alumina was contacted with 10 ml of a aqueous solution of LiNO₃. The weight of lithium added in the pore impregnation step was double the amount of lithium as present in the final catalyst composition (100 wt % excess of alkaline metal).

The solids were subsequently dried in a stove at 105° C. for 16 hours and subsequently subjected to a calcination at 350° C. for 2 hours to obtain the respective cerium and/or lithium oxides on the surface of the alumina.

The thus obtained loaded alumina was used as support for the Au particles. The prepared mixed oxides had an intended Ce/Al and Li/Al molar ratio of 1/15.

The gold catalysts were prepared via homogeneous deposition precipitation using urea as precipitating agent. The loaded alumina was first suspended in 25 ml of Millipore water (18.2 MSΩ cm resistive Milli-Q water). To this suspension 25 ml of an aqueous solution of HAuCl₄.3aq (99.999% Aldrich chemicals) was added. The intended Au/Al molar ratio was 1/75. This ratio of 1:75 is equal to 5 wt % Au. The temperature was kept at 80° C. allowing urea (p.a., obtained from Acros) to decompose ensuring a slow increase in pH. When a pH of around 8-8.5 was reached, the slurry was filtrated and washed thoroughly with ultra pure (18.2 MΩ cm resistive Milli-Q water.) water until no Cl was detected in the filtrate. The chlorine concentration was tested by titration with AgNO₃. The catalyst was dried overnight at 80° C. The catalysts were thoroughly ground to ensure that the macroscopic particle size was around 200 μm. No calcination was applied to the catalyst.

The gold and Ce and Li concentrations were determined by Inductively Coupled Plasma Optical Emission Spectroscopy (ICP-OES) using a Varian Vista-MPX. For that purpose, a small fraction of the catalyst was dissolved in diluted aqua regia.

X-ray diffraction measurements were taken using a Philips Goniometer PW 1050/25 diffractometer equipped with a PW Cu 2103/00 X-ray tube operating at 50 kV an 40 mA. The average particle size was estimated from XRD line broadening after subtraction of the signal from the corresponding support by using the Scherrer equation as described in P. Scherrer, Nadu. K. Ges. Wiss (1918) 98. [32] A. C. Gluhoi, N. Bogdanchikova, B. E. Nieuwenhuys, J. Catal. 229 (2005) 159. The average gold particle size of the catalysts could not be determined by XRD because the size of the particles was below the detection limit of 3 nm. The presence of such small nano-particles were confirmed using High Resolution TEM.

The total surface area was determined by N₂ adsorption using a Qsurf M1 analyzer (Thermo Finnigan).

The properties of the catalyst thus obtained is listed in Table 1.

TABLE 1

| | | | | Au—Li₂O/ |
|---|---|---|---|---|
| catalyst | Au | Au—CeOₓ | Au—Li₂O | CeOx |
| Metal loading | 4.6 ± 0.1 | 4.1 ± 0.1 | 4.5 ± 0.3 | 4.0 ± 0.2 |
| (wt %) | | | | |
| Average gold | 4.3 ± 0.1 | <3.0 | 3.2 ± 0.1 | <3.0 |
| particle size (nm) | | | | |
| Total surface area | 260 ± 5 | 218 ± 7 | 278 ± 7 | 262 ± 7 |
| SBET (m²/g) | | | | |

### Example 2

Example 1 was repeated except that instead of gold a copper comprising catalyst was prepared using Cu(NO₃)₂.3aq.

The weight of lithium added in the pore impregnation step was double the amount of lithium as present in the final catalyst composition.

The properties of the catalyst thus obtained are listed in Table 2.

TABLE 2

| Catalyst | Cu | Cu—CeOₓ | Cu—Li₂O |
|---|---|---|---|
| Metal loading | 1.5 ± 0.1 | 1.0 ± 0.1 | 1.4 ± 0.1 |
| (wt %) | | | |
| Average copper | 3.5 ± 0.1 | <3.0 | <3.0 |
| particle size (nm) | | | |
| Total surface area | 259 ± 10 | 253 ± 10 | 268 ± 10 |
| SBET (m²/g) | | | |

### Example 3

Example 1 was repeated except that instead of gold a silver comprising catalyst was prepared using AgNO₃.

The weight of lithium added in the pore impregnation step was double the amount of lithium as present in the final catalyst composition.

Because urea and silver atoms can form a soluble Ag[NH₃]₂⁺ complex, a large surplus of silver was needed to deposit enough silver on the Al₂O₃. The aqueous solution used in step 2 contained AgNO₃ in a concentration of 2 g/l.

The properties of the catalyst thus obtained is listed in Table 3.

TABLE 3

| Catalyst | Ag—CeOₓ | Ag—Li₂O |
|---|---|---|
| Metal loading | 1.7 ± 0.1 | 2.2 ± 0.1 |
| (wt %) | | |
| Average silver | <3.0 | <3.0 |
| particle size (nm) | | |
| Total surface area | 260 ± 10 | 270 ± 10 |
| SBET (m²/g) | | |

### Example 4

### Ethanol Oxidation in an EtOH/O₂ Mixture of 1

The activity of the catalysts were measured in a microreactor system. Oxygen flow balanced in argon was bubbled through a vessel containing absolute ethanol. This gas flow was led to a lab-scale flow reactor made from quartz with an internal diameter of 1 cm. In the reactor, the catalyst was placed on a quartz bed. The amount of catalyst used was 0.3 g for the Au—CeOₓ catalyst. For the Au—Li₂O, the amount of catalyst was adjusted in such a way that the amount of gold was similar as for the Au—CeOₓ catalyst. Prior to the activity experiments, the catalysts were reduced with H₂ (4 vol % in Ar) at 400° C. for 2 hours.

The oxygen/ethanol as used as feed had an oxygen: ethanol molar ratio of 1:1. Ethanol used consisted of 96 vol. % ethanol and 4 vol. % water. In the experiments a total gas flow of 40 ml⁻¹ (GHSV ˜2500 h⁻¹) was maintained. The effluent stream was analyzed on-line by a gas chromatograph (HP 8590) with a CTR1 column (Alltech) containing a porous polymer mixture, an activated molecular sieve and a Hayesep Q column (Alltech). All possible reaction products were calibrated by injecting a dilute solution directly into the GC or in case of gases as ethylene and ethylene oxide, the gas flow from lecture bottles was diluted with argon and led to the GC. Mass spectrometry confirmed that the analysis of the reaction products by gas chromatography was correct. To distinguish the different components, the relative intensity ratios of masses 15, 29, 43, 44, 45 were used. The experiments were carried out at atmospheric pressure. Each reaction test consisted of at least two heating-cooling cycles from room temperature up to 400° C., with a rate of 2° C./min in order to monitor possible catalyst deactivation and hysteresis processes.

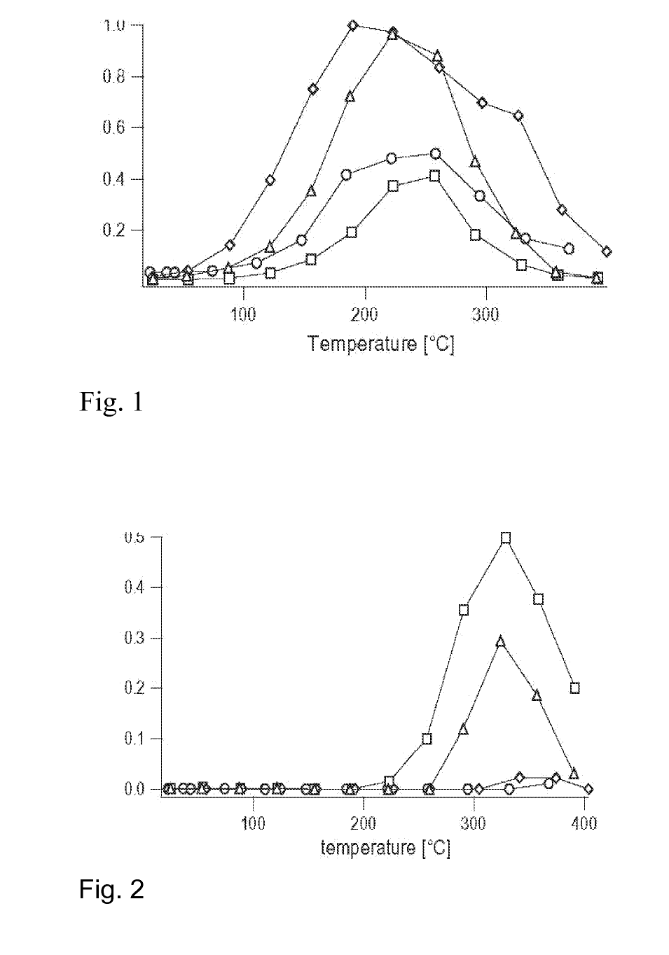

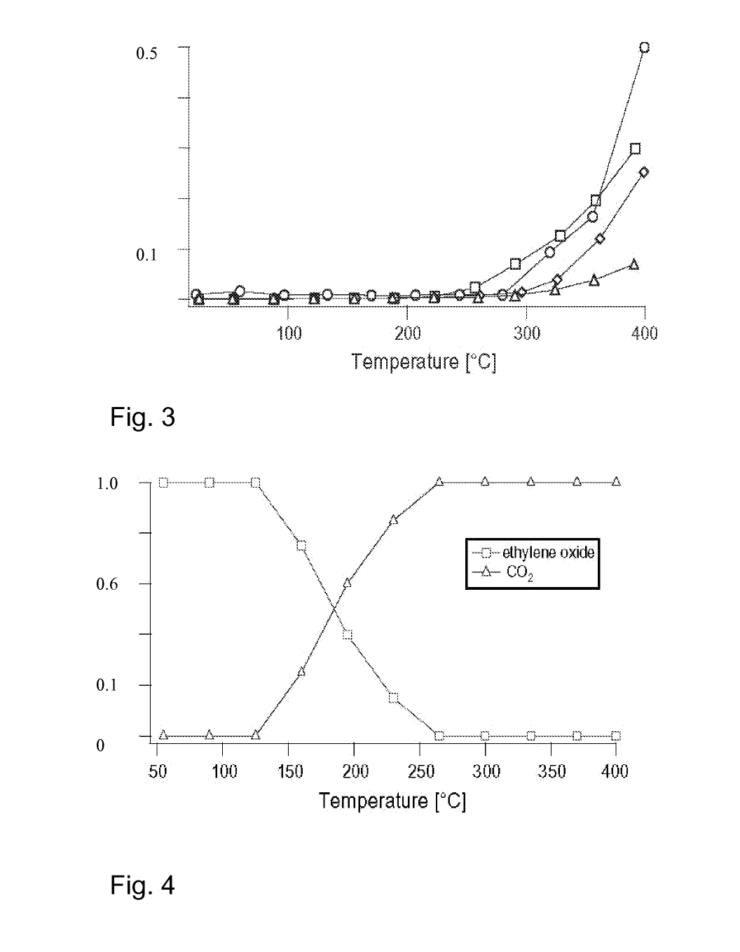

In the first heating cycle the reaction starts at higher temperatures compared to the cooling step. In the subsequent cycles, the behaviour is rather similar to that of the first cooling step. The conversion starts at 100° C. and reaches a maximum at about 275° C. The Au/Li₂O/Al₂O₃ shows the best activity. The oxygen conversion starts at higher temperatures compared to the ethanol conversion. The presence of Li₂O or CeOₓ lowers the temperature of oxygen uptake by 50° C. The oxygen conversion starts at 150° C. and reaches a maximum conversion at 250° C. for the CeOₓ containing catalysts, and for the Au/Li₂O/Al₂O₃, the oxygen conversion reaches maximum conversion at 350° C. At temperatures between 100° C. and 250° C., the main product is ethylene oxide. This is illustrated in FIG. 1 which shows the selectivity to ethylene oxide at various temperatures for the different catalysts. The open circles represent the results for the Au catalyst of Example 1 not containing a CeOₓ or Li₂O additive, the open diamonds represent the results for the Au—Li₂O catalyst of Example 1, the open boxes represent the results for the Au—CeOₓ catalyst of Example 1 and the triangles represent the Au—Li₂O/CeOₓ catalyst of Example 1. As can be seen in FIG. 1 the catalyst with the best performance in ethylene oxide formation is Au—Li₂O catalyst. A selectivity to ethylene oxide of 88% is achieved. With this catalyst, also traces of the combination product of ethylene oxide and ethanol (ethoxy-ethanol) were detected. When the gas flow was bubbled through a diluted NaOH solution, glycol was produced, which is further evidence that the output gas flow contained ethylene oxide. At temperatures between 250 and 400° C., diethyl ether was formed over the two CeOₓ-containing catalysts, as shown in FIG. 2. FIG. 2 shows the selectivity to diethyl ether at various temperatures for the different catalysts. The addition of ceria to the Au/Al₂O₃ catalyst also results in more ethane formation (not shown). Also, ethylene and CO₂ and traces of CO were formed as shown in FIG. 3 for ethylene.

### Example 5

### Ethanol Oxidation in an EtOH/O₂ Mixture of 6

Example 4 is repeated except that the ethanol: oxygen molar ratio was 1:6. The results of ethanol oxidation over the Au—Li₂O catalyst of Example 1 is in excess oxygen (molar ethanol/O₂=1/6) is presented in FIG. 4. It has been observed that ethanol starts to convert at 150° C. and a sharp increase in conversion is observed at 200° C. At this temperature, also the O₂ conversion and the CO₂ production start. At temperatures above 300° C., ethanol is mainly oxidized to CO₂. The ethylene oxide production can be assigned to the activity of gold as the c-Al₂O₃ support produces no ethylene oxide. Addition of Li₂O has shown to increases the ethanol conversion between 50 and 200° C. as compared to the catalysts not containing an additive or containing a CeOₓ additive. The catalyst comprising Li₂O and not containing CeOₓ also showed a better activity at lower temperatures than the Au—Li₂O/CeOₓ catalyst. The main product in this temperature region of 50 to 200° C. is ethylene oxide, while no oxygen is consumed.

### Example 6

Example 4 was repeated using the Ag—Li₂O catalyst of Example 3 and the Cu—Li₂O catalyst of Example 2. The results at various temperatures are presented in Table 4.

TABLE 4

| | | | Selectivity | Selectivity | Selectivity |
|---|---|---|---|---|---|
| | Temper- | Ethanol | to ethylene | to di-ethyl | to |
| | ature | conversion | oxide | ether | ethylene |
| Catalyst | (° C.) | (%) | (mol %) * | (mol %) | (mol %) |
| Au—Li₂O | 200 | 80 | 95 | 0 | 0 |
| | 300 | 90 | 71 | 0 | 2 |
| | 400 | 100 | 10 | 3 | 26 |
| Ag—Li₂O | 200 | 58 | 96 | 0 | 0 |
| | 300 | 90 | 54 | 21 | 23 |
| | 400 | 100 | 30 | 38 | 28 |
| Cu—Li₂O | 200 | 70 | 90 | 0 | 0 |
| | 300 | 92 | 15 | 12 | 60 |
| | 400 | 100 | 4 | 2 | 90 |

### Example 7

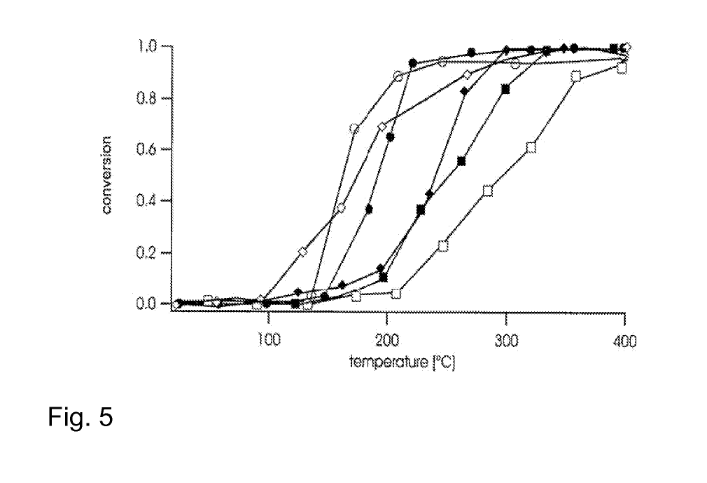

Example 4 was repeated with the copper-based catalysts as prepared in Example 2. The selectivities (expressed in mol %) to ethylene, acetaldehyde, diethyl ether, CO and ethylene oxide represented in Table 5. The ethanol conversion in the first heating stage and in the cooling stage is shown in FIG. 5.

The circles represent the results for the Cu catalyst not containing a CeOₓ or Li₂O additive, the diamonds represent the results for the Cu—Li₂O catalyst of Example 2, the boxes represent the results represent the results for the Cu—CeOₓ catalyst. The closed symbols are the results for the first heating stage. The closed symbols are the results for the cooling stage.

This example illustrates that a process wherein the copper catalyst with and without the additive is used can prepare ethylene in a high yield at a temperature between 350 and 450° C. A process with a copper-catalyst not containing the additive shows the highest yield to ethylene.

TABLE 5

| | Temperature | | acetal- | Diethyl | | Ethylene |
|---|---|---|---|---|---|---|
| Catalyst | (° C.) | ethylene | dehyde | ether | CO | oxide |
| Cu | 200 | 0 | 0 | 0 | 30 | 70 |
| | 300 | 15 | 15 | 0 | 20 | 50 |
| | 400 | 100 | 0 | 0 | 0 | 0 |
| Cu—CeOₓ | 200 | 0 | 0 | 0 | 0 | 0 |
| | 300 | 20 | 5 | 0 | 55 | 20 |
| | 400 | 60 | 0 | 0 | 40 | 0 |
| Cu—Li₂O | 200 | 0 | 0 | 0 | 10 | 90 |
| | 300 | 60 | 12 | 12 | 0 | 15 |
| | 400 | 90 | 2 | 2 | 2 | 4 |

That which is claimed:

1. Process to prepare an ethanol-derivate compound or compounds by reacting ethanol in the presence of molecular oxygen and a catalyst comprising a gamma-alumina carrier, metal nano-particles wherein the metal is selected from silver, copper or gold.

2. Process according to any claim 1, wherein the catalyst comprises an additive selected from the group of a cerium compound or an alkaline metal compound.

3. Process according to claim 2, wherein the additive is an alkaline metal compound selected from the group consisting of Na, Li or K.

4. Process according to claim 3, wherein the alkaline metal compound is Li and wherein the Li metal is present in the catalyst as an oxide or hydroxide.

5. Process according to claim 1, wherein the gamma-alumina carrier used to prepare the catalyst comprised between 0.05 and 0.2 wt % of sodium oxide and between 0.01 and 0.1 wt % of an iron oxide.

6. Process according to claim 1, wherein the metal nano-particles have an average size of less than 5 nm as determined by XRD.

7. Process according to claim 1, wherein the nano-particles are gold nano-particles.

8. Process according to claim 1, wherein the prepared ethanol-derivative is ethylene and wherein the nano-particles are copper nano-particles.

9. Process according to claim 8, wherein the temperature is between 350 and 450° C.

10. Process according to claim 1, wherein the ethanol-derivative compounds are ethylene oxide, diethyl ether and/or ethylene.

11. Process according to claim 1, wherein the molar ratio of ethanol and molecular oxygen is between 1:0.5 and 1:10.

12. Process according to claim 1, wherein the pressure at which the process is performed is between 0.1 and 1 MPa and the gas hourly space velocity (GHSV) is in the range of from 500 to 5000 h⁻¹.

13. Process according to claim 12, wherein the catalyst comprises nano-particles of an average size of less than 5 nm, the additive is Li₂O and the temperature is between 100 and 250° C.

14. Process according to claim 1, wherein the process is performed in a reactor comprising the catalyst, to which reactor a gaseous feed comprising ethanol, oxygen and an optional diluting gas is supplied and from which reactor an effluent is discharged comprising the ethanol-derivative compound or compounds, oxygen and the optional diluting gas.

15. Process according to claim 14, wherein the reactor is a fluidized bed reactor.

16. Process according to claim 14, wherein the reactor is a packed bed reactor.

17. Process according to claim 14, wherein from the effluent any non-converted ethanol is separated from the reactor effluent and recycled to the feed comprises less than 10 vol % water.

18. Process according to claim 17, wherein ethanol is separated from the reactor effluent by means of distillation.

19. Process according to claim 14, wherein carbon dioxide as present in the reactor effluent is recycled to the reactor to act as diluent for the ethanol feed.

20. Process to prepare ethylene glycol, an ethylene glycol ether or an ethanol amine from ethanol by first preparing ethylene oxide according to the process according to claim 10 and converting ethylene oxide as obtained into the desired ethylene glycol, an ethylene glycol ether or an ethanol amine.

21. Process to prepare a catalyst composition comprising a gamma-alumina carrier, metal nano-particles, wherein the metal is selected from silver, copper or gold, and an additive selected from the group of an alkaline metal compound as present as an oxide or hydroxide of the alkaline metal wherein:
in a first catalyst preparation step a gamma-alumina carrier is contacted with an aqueous solution comprising a salt of the alkaline metal in an impregnation step to obtain a loaded alumina carrier, and wherein the weight of alkaline metal as deposited on the alumina surface of the alumina carrier is greater than the weight of alkaline metal as present in the final catalyst composition, drying the loaded alumina carrier and subjecting the dried loaded alumina carrier to a calcination step,
loading the silver, copper or gold metal to the calcined loaded carrier in a second catalyst preparation step by contacting with an aqueous solution comprising a silver, copper or gold metal salt and drying to obtain the catalyst composition.

22. Process to prepare a catalyst composition comprising a gamma-alumina carrier, metal nano-particles wherein the metal is selected from silver, copper or gold and an additive selected from the group of cerium compound or an alkaline metal compound as present as an oxide or hydroxide of the cerium or alkaline metal wherein:
in a first catalyst preparation step a gamma-alumina carrier is contacted with an aqueous solution comprising a salt of the cerium or alkaline metal in an impregnation step to obtain a loaded alumina carrier, drying the loaded alumina carrier and subjecting the dried loaded alumina carrier to a calcination step,
and loading the silver, copper or gold metal to the calcined loaded carrier in a second catalyst preparation step by contacting with an aqueous solution of a silver, copper or gold metal salt and drying to obtain the catalyst composition, wherein
the gamma-alumina carrier used to prepare the catalyst in the first catalyst preparation step has a dried surface which is expressed by its iso-electric point of greater than 7 as measured by preparing an aqueous slurry of the alumina at ambient conditions and measuring the pH of the water phase, wherein the iso-electric point is the measured pH.
